(19) United States
(12) Patent Application Publication
(10) Pub. No.: US 2001/0031269 A1
(43) Pub. Date: Oct. 18, 2001

(54) Transfer-resistant cosmetic composition comprising a non-volatile silicone compound and a non-volatile hydrocarbon-based oil which is incompatible with this silicone compound

(75) Inventor: Pascal Arnaud, L'Hay Les Roses (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'Oreal, Paris (FR)

(21) Appl. No.: 09/728,056

(22) Filed: Dec. 4, 2000

(30) Foreign Application Priority Data
Dec. 3, 1999 (FR) 9915373

(51) Int. Cl. A61K 6/00; A61K 7/00

(52) U.S. Cl. 424/401

(57) ABSTRACT

The present invention provides a composition, which includes:

at least one volatile hydrocarbon-based solvent;

at least one non-volatile silicone compound which is soluble or dispersible in the volatile hydrocarbon-based solvent; and

at least one non-volatile hydrocarbon-based oil which is soluble in the volatile hydrocarbon-based solvent and incompatible with the non-volatile silicone compound;

wherein the non-volatile hydrocarbon-based oil has solubility parameters satisfying the relationships, 16.40 (J/cm³)^{½}≦δ_{D}≦19.00 (J/cm³)^{½} and 2.00 (J/cm³)^{½}≦δₐ≦9.08 (J/cm³)^{½}. Other embodiments of the present invention provides methods of making and using the above-noted composition, as well as compositions that include the above-noted composition. By the present invention, it is possible to obtain a glossy deposit having very good staying power, which undergoes little or no transfer, does not migrate and is water-resistant, while at the same time being very pleasant to apply and to wear throughout the day.

### BACKGROUND OF THE INVENTION

1. Field of the Invention

The present invention relates to a composition containing a non-volatile silicone compound and a non-volatile hydrocarbon-based oil that are mutually incompatible, which is intended in particular for the cosmetics field. More preferably, the invention relates to a glossy transfer-resistant composition to care for and/or make up the skin, including both human facial and body skin, the lips, the upper and lower eyelids, or superficial body growths such as the eyelashes, the eyebrows, the nails and the hair.

This composition is preferably in the form of a product cast as a stick or as a dish, for instance lipsticks or lip balms, cast foundations, concealer products, eyeshadows or blushers, in the form of a more or less fluid paste or cream, for instance fluid foundations or lipsticks, eyeliners, mascaras, antisun compositions, colouring compositions or artificial tanning compositions for the skin or alternatively make-up compositions for the body or the hair.

2. Discussion of the Background

Make-up or care products for human skin or lips, for instance foundations or lipsticks, generally contain fatty phases such as waxes and oils, pigments and/or fillers and optionally additives such as cosmetic or dermatological active agents. They can also contain so-called “pasty” products of soft consistency, which make it possible to obtain colored or noncolored pastes that can be applied with a brush.

When the above-mentioned compositions are applied to the skin or the lips, they have the drawback of transferring, i.e., of becoming at least partly deposited on or leaving traces on certain supports with which they may come into contact, such as glass, a cup, cigarettes, an item of clothing or the skin. This results in mediocre persistence of the applied film, making it necessary to reapply the foundation or lipstick composition regularly. Moreover, the appearance of these unacceptable traces or deposits, especially on shirt collars, can put certain women off using this type of make-up. Furthermore, these compositions have a tendency to migrate, i.e., to travel in the wrinkles and fine lines of the skin around the lips and the eyes, resulting in an unaesthetic effect.

In WO-A-96/40044, the company Procter & Gamble envisaged lipstick compositions having transfer-resistance properties, containing a volatile oil and a non-volatile oil of the perfluoropolyether type, which are incompatible. WO-A-96/40044 also discloses the enhancement of gloss by means of the prior dispersion of an oily phase in a matrix, and the ability of this oily phase to segregate during the application of the product to the support and to migrate to the surface of the film thus deposited. However, this system requires good dispersion of the oily phase in the matrix and can give rise to problems of stability of the product that are associated with the obligatory poor compatibility of the oily phase with the matrix.

It is known that the enhancement of the gloss properties requires good dispersion of the solid particles in the composition, and particularly the good dispersion of pigments. U.S. Pat. No. 5,945,092 from Revlon thus discloses the use of silicone surfactants combined with volatile oils. Despite their efficacy, however, these surfactants have the drawback of being potentially irritating, and particularly to labial mucous membranes, when these surfactants are present in the composition in large amounts (typically greater than 3%).

It is thus particularly advantageous to find another means for improving the gloss of transfer-resistant compositions without incurring the drawbacks mentioned above.

In addition, although the above-described compositions have improved “transfer-resistance” properties, they have the drawback of leaving on the lips, after the volatile oils have evaporated, a film which very quickly becomes uncomfortable over time (sensation of dryness, tautness and discomfort), which puts a certain number of women off this type of lipstick. Furthermore, the resulting deposit is matt. Nowadays, consumers are seeking a glossy product, which is comfortable to wear throughout the day, which undergoes little or no migration in the folds of skin around the lips or the eyes, and which undergoes no or virtually no transfer.

### SUMMARY OF THE INVENTION

It is an object of the present invention to improve the gloss of transfer-resistant compositions without incurring the drawbacks mentioned above.

It is another object of the present invention to provide a glossy product, which is comfortable to wear throughout the day.

It is another object of the present invention to provide a glossy product, which undergoes little or no migration in the folds of skin around the lips or the eyes.

It is another object of the present invention to provide a glossy product, which undergoes no or virtually no transfer.

It is another object of the present invention to provide a composition which does not have the drawbacks of the conventional compositions and which has good “transfer-resistance” and migration-resistance properties, even under a pronounced pressure or rubbing.

It is another object of the present invention to provide a composition, which meets the consumer's desires, does not dry out and does not pull the skin or the lips on which it is applied, either during the application or over time.

These and other objects may be accomplished with the present invention, the first embodiment of which provides a composition, which includes:

at least one volatile hydrocarbon-based solvent;

at least one non-volatile silicone compound which is soluble or dispersible in the volatile hydrocarbon-based solvent; and

at least one non-volatile hydrocarbon-based oil which is soluble in the volatile hydrocarbon-based solvent and incompatible with the non-volatile silicone compound;

wherein the non-volatile hydrocarbon-based oil has solubility parameters satisfying the relationships, 16.40 (J/cm³)^{½}>δ_{D}≦19.00 (J/cm³)^{½} and 2.00 (J/cm³)^{½}≦δₐ<9.08 (J/cm³)^{½}.

Another embodiment of the present invention provides a composition selected from the group including lipstick composition, cosmetic composition, transfer-resistant composition, glossy composition, composition for topical application, and combinations thereof, which includes:

at least one volatile hydrocarbon-based solvent;

at least one non-volatile silicone compound which is soluble or dispersible in the volatile hydrocarbon-based solvent; and

at least one non-volatile hydrocarbon-based oil which is soluble in the volatile hydrocarbon-based solvent and incompatible with the non-volatile silicone compound;

wherein the non-volatile hydrocarbon-based oil has solubility parameters satisfying the relationships, 16.40 (J/cm³)^{½}≦δ^{D}≦19.00 (J/cm³)^{½} and 2.00 (J/cm³)^{½}≦δₐ≦9.08 (J/cm³)^{½}.

Another embodiment of the present invention provides a method for preparing a composition, which includes:

contacting

at least one volatile hydrocarbon-based solvent;

at least one non-volatile silicone compound which is soluble or dispersible in the volatile hydrocarbon-based solvent; and

at least one non-volatile hydrocarbon-based oil which is soluble in the volatile hydrocarbon-based solvent and incompatible with the non-volatile silicone compound;

wherein the non-volatile hydrocarbon-based oil has solubility parameters satisfying the relationships, 16.40 (J/cm³)^{½}≦δ_{D}≦19.00 (J/cm³)^{½} and 2.00 (J/cm³)^{½}≦δₐ<9.08 (J/cm³)^{½}.

Another embodiment of the present invention provides a method of use, which includes applying any of the above-described compositions according to the invention to the lips or the skin, respectively.

Another embodiment of the present invention provides a process selected from the group including reducing or preventing the transfer of a film of composition deposited on the skin and/or the lips of a human being to a support placed in contact with the film, preserving the gloss of a film of a composition deposited on the skin and/or lips of a human being, reducing or preventing the transfer of a care or make-up composition for the skin or the lips onto a support other than the skin or the lips, and combinations thereof, the composition including at least one ingredient selected from the group including cosmetic or dermatological active agents, dyestuffs, and combinations thereof, which process includes introducing into the composition:

at least one volatile hydrocarbon-based solvent;

at least one non-volatile silicone compound which is soluble or dispersible in the volatile hydrocarbon-based solvent; and

at least one non-volatile hydrocarbon-based oil which is soluble in the volatile hydrocarbon-based solvent and incompatible with the non-volatile silicone compound; wherein the non-volatile hydrocarbon-based oil has solubility parameters satisfying the relationships, 16.40 (J/cm³)^{½}≦δ_{D}≦19.00 (J/cm³)^{½} and 2.00 (J/cm³)^{½}≦δₐ≦9.08 (J/cm³)^{½}.

By the present invention, it is possible to obtain a glossy deposit having very good staying power, which undergoes little or no transfer, does not migrate and is water-resistant, while at the same time being very pleasant to apply and to wear throughout the day.

### DESCRIPTION OF THE PREFERRED EMBODIMENTS

Various other objects, features and attendant advantages of the present invention will be more fully appreciated as the same becomes better understood from the following detailed description of the preferred embodiments of the invention.

The present inventor has found that the use of a non-volatile silicone compound, a non-volatile hydrocarbon-based oil, which is incompatible with the non-volatile silicone compound, and a specific volatile solvent, in a physiologically acceptable composition and more preferably a cosmetic composition, makes it possible to obtain a glossy deposit of very good staying power, which undergoes little or no transfer, does not migrate and is water-resistant, while at the same time being very pleasant to apply and to wear throughout the day. The deposit is soft and creamy.

One preferred embodiment of the present invention is thus a care or make-up composition for keratin materials, including at least one volatile hydrocarbon-based solvent, at least one non-volatile silicone compound which is soluble or dispersible in the volatile hydrocarbon-based solvent, and at least one nonvolatile hydrocarbon-based oil which is soluble in the volatile hydrocarbon-based solvent and incompatible with the non-volatile silicone compound, the nonvolatile hydrocarbon-based oil having solubility parameters such that 16.40 (J/cm³)^{½}≦δ_{D}≦19.00 (J/cm³)^{½} and 2.00 (J/cm³)^{½}≦δ_{α}≦9.08 (J/cm³)^{½}.

The term “oil” means any non-aqueous medium which is liquid at room temperature (25° C.) and atmospheric pressure (760 mm Hg). The term “solvent” means a non-aqueous medium which is liquid at room temperature (25° C.) and atmospheric pressure (760 mm Hg). The term “volatile” means a medium which can evaporate from the skin or the lips in less than one hour. A volatile medium is chosen in particular from media which have a vapor pressure, at room temperature and atmospheric pressure, ranging from 10⁻³ mm Hg to 300 mm Hg (0.13 Pa to 40,000 Pa).

Thus, the composition can contain one or more non-volatile hydrocarbon-based oils and one or more volatile hydrocarbon-based solvents.

This composition is preferably a cosmetic or dermatological composition. It may thus contain ingredients that are compatible with the skin, the lips, keratin fibres and the nails. It can be in the form of an anhydrous gel, an oil-in-water or water in-oil emulsion or dispersion, or alternatively in the form of a multiple emulsion. It can also be in a more or less fluid form, in the form of a paste or in the form of a non-deformable or rigid solid, optionally cast as a stick or a dish. It is preferably in fluid or stick form, in particular anhydrous fluid or stick form. The term “fluids means a composition which flows under its own weight, as opposed to a solid.

According to the invention, the non-volatile silicone compound and the non-volatile hydrocarbon based oil are mutually incompatible, in the absence of volatile solvent. After depositing the composition on the keratin materials, the volatile solvent evaporates, in particular leaving on these materials the nonvolatile silicone compound, the non-volatile hydrocarbon-based oil and, if present, the particulate filler. The deposit obtained on the skin or the lips after drying is homogeneous, glossy and soft. It leaves virtually no traces on a support which comes into contact with the deposit and does not migrate, especially in the fine lines around the lips.

Preferably, the term, “incompatible” means insoluble or immiscible.

The composition preferably contains at least one ingredient chosen from dyestuffs and cosmetic and dermatological active agents, and mixtures thereof. By virtue of the incompatibility of the non-volatile silicone compound and of the non-volatile hydrocarbon based oil, the composition of the invention makes it possible to limit or prevent altogether the transfer of the composition and in particular the transfer of the active agents and/or dyestuffs and thus to keep these active agents and/or dyestuffs where they were deposited.

Another preferred embodiment of the invention is the use, in a cosmetic composition or for the manufacture of a composition for topical application, of a combination of at least one volatile hydrocarbon-based solvent, of at least one non-volatile silicone compound which is soluble or dispersible in the volatile solvent and of at least one non-volatile hydrocarbon-based oil which is soluble in the volatile solvent and incompatible with the non-volatile silicone compound, the nonvolatile hydrocarbon-based oil having solubility parameters such that 16.40 (J/cm³)^{½}≦δ_{D}≦19.00 (J/cm³)^{½} and 2.00 (J/cm³)^{½}≦δₐ≦9.08 (J/cm³)^{½} as an agent for reducing or even preventing altogether the transfer of a film of composition deposited on the skin and/or the lips of a human being onto a support placed in contact with the film and/or for preserving its gloss.

Another preferred embodiment of the invention is the use, in a cosmetic composition or for the manufacture of a composition for topical application, of a combination of at least one volatile hydrocarbon-based solvent, of at least one non-volatile silicone compound which is soluble or dispersible in the volatile solvent, and of at least one non-volatile hydrocarbon-based oil which is soluble in the volatile solvent and incompatible with the non-volatile silicone compound, the nonvolatile hydrocarbon-based oil having solubility parameters Such that 16.40 (J/cm³)^{½}≦δ_{D}≦19.00 (J/cm³)^{½} and 2.00 (J/cm³)^{½}≦δₐ≦9.08 (J/cm³)^{½} as an agent, the said composition being transfer-resistant and/or glossy.

Another preferred embodiment of the invention is a cosmetic process to care for or make up the lips, superficial body growths or the skin, which includes applying to the lips, superficial body growths or the skin, respectively, a cosmetic composition as defined above.

Another preferred embodiment of the invention is a process for limiting or even preventing altogether the transfer and/or migration of a care or make-up composition for the skin or the lips onto a support other than the said skin and the said lips, containing at least one ingredient chosen from dyestuffs and cosmetic and dermatological active agents, which includes introducing into the composition a system including at least one non-volatile silicone compound, at least one non-volatile hydrocarbon-based oil which is incompatible with the non-volatile silicone compound, and at least one volatile oil which is a solvent for the non-volatile silicone compound and for the non-volatile hydrocarbon-based oil, as defined above.

The composition according to the invention has particularly advantageous spreading and adhesion qualities on the skin and the lips, as well as a pleasant, creamy feel. This composition also has the advantage of being easy to remove, especially with a conventional make-up removing milk. This is entirely noteworthy since the compositions of the prior art with high “transfer resistance” properties are very difficult to remove. In general, they are sold with a specific make-up removing product, which places an additional constraint on the user.

The composition according to the invention thus advantageously includes one or more physiologically acceptable non-volatile silicone compounds.

The non-volatile silicone compounds of the invention must be soluble or dispersible in volatile hydrocarbon-based solvents and more preferably in volatile isoalkanes. They are preferably chosen from compounds that are liquid at room temperature and mixtures thereof and, even more preferably, they have a viscosity which is within the range from 5 to 10,000 cSt at 25° C. and better still from 10 to 5,000 cSt. These ranges include all values and subranges therebetween, including 15, 25, 35, 45, 85, 100, 150, 300, 900, 1200, 1800, 3300, 4500, 6500, 7200, 8100 and 9000 cSt.

Examples which may be mentioned are polydimethylsiloxanes, phenyltrimethicones, polyalkylmethylsiloxanes, silicone resins such as those disclosed in documents JP-A-62 61911, JP-A-61 65809 and EP-A-602 905, and fluorosilicones, and mixtures thereof. The entire contents of each of the aforementioned references is hereby incorporated by reference.

These silicone compounds are preferably chosen from non-volatile polydimethylsiloxanes (PDMSs); polydimethylsiloxanes including alkyl, alkoxy or phenyl groups pendant or at the end of a silicone chain, these groups containing from 2 to 24 carbon atoms, more preferably 4 to 20 carbon atoms, and most preferably 8 to 18 carbon atoms (which ranges are inclusive of all values and subranges therebetween, including 3, 5, 7, 9, 10, 11, 12, 13, 14, 15, 16, 17, 19, 21, 22 and 23 carbon atoms); phenyl trimethicones, phenyl dimethicones, phenyl trimethylsiloxydiphenylsiloxanes, diphenyl dimethicones, diphenyl methyldiphenyltrisiloxanes, 2-phenylethyl trimethylsiloxysilicates; fluorosilicones including a fluoro group pendant or at the end of a silicone chain and containing from 1 to 12 carbon atoms, more preferably from 2 to 10 carbon atoms, and most preferably from 4 to 6 carbon atoms (which ranges are inclusive of all values and subranges therebetween, including 2, 3, 5, 7, 9, 10, and 11 carbon atoms), all or some of the hydrogen atoms of which are substituted with fluorine atoms; silicone resins, and mixtures thereof.

Their content by mass in the final composition is, for example, within the range from 0.5% to 90% and preferably from 5% to 60% and even more preferably from 10% to 50%. These ranges include all values and subranges therebetween, including 0.75, 1, 2, 3, 4, 6, 12, 15, 18, 20, 25, 35, 45, 55, 65, 75, 85 and 90%).

The non-volatile silicone compound is preferably in a proportion equal to or greater than that of the non-volatile hydrocarbon-based oil, in other words the ratio R defined by:

$$R = \frac{\text{\% by mass of non-volatile silicone compound}}{\text{\% by mass of non-volatile hydrocarbon-based oil}}$$

is preferably greater than or equal to 1. More preferably, the ratio R is greater than or equal 5 to 2, most preferably greater than or equal to 3, and most particularly preferably, greater than or equal to 4. These ranges include all values and subranges therebetween, including 1.25, 1.5, 1.75, 2.5, 3.5, 5, 10, and 15.

Preferably, the non-volatile hydrocarbon-based oils of the invention should also be soluble in the volatile hydrocarbon-based solvents, for instance volatile isoalkanes, but, on 10 the other hand, they preferably should not be soluble in the silicone compounds described above.

Preferably, these hydrocarbon-based oils are fluid at room temperature and include in their chemical structure at least one nonionic polar group below:

COOH

monosubstituted or disubstituted OH (primary or secondary)

NHR wherein R represents H or a linear or branched C₁ to C₂₀ alkyl or alkoxy group; NR₁R₂ wherein R₁ and R₂ optionally forming a ring and each independently representing a linear or branched C₁ to C₂₀ alkyl or alkoxy radical or group, or

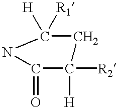

with R1′ and R2′ each independently representing H or a linear or branched C1 to C20 alkyl or alkoxy group, and preferably at least two nonionic polar groups below:

COOH

monosubstituted or disubstituted OH (primary or secondary)

PO₄

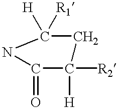

with R1′ and R2′ each independently representing H or a linear or branched C₁ to C₂₀ alkyl or alkoxy group. The hydrocarbon-based oils advantageously include at least one OH group. The C₁ to C₂₀ range for the alkyl or alkoxy groups and radicals above include all values and subranges therebetween, including 2, 3, 4, 5, 6, 7, 8, 9, 10, 11, 12, 13, 14, 15, 16, 17, 18 and 19 carbons.

The non-volatile hydrocarbon-based oils according to the invention are preferably such that their Hansen solubility parameters δ_{D}, δₚ and δₕ are such that: 16.40 (J/cm³)_{½}≦δ_{D}≦19.00 (J/cm³)_{½} and preferably 16.70 (J/cm³)^{½}≦δ_{D}≦18.50 (J/cm³)^{½}. 2.00 (J/cm³)^{½}≦δₐ≦9.08 (J/cm³)^{½} and preferably 4.00 (J/cm³)^{½}≦δₐ≦9.08 (J/cm³)^{½} and better still 5.00 (J/cm³)^{½}≦δₐ≦9.08 (J/cm³)^{½} and better still 5.00 (J/cm³)^{½}≦δₐ≦6.80 (J/cm³)^{½} given that δₐ=(δₚ²+δₕ²)^{½}. These ranges include all values and subranges therebetween, including (for δ_{D}) 16.50, 16.60, 16.80, 17.00, 17.50, 17.70, 18.00 and 18.20; (for δₐ) 2.20, 2.40, 3.00, 3.50, 3.80, 4.20, 4.40, 5.30, 5.80, 6.20, 6.40, 7.00, 7.20, 7.60, 8.10, and 9.00.

The definition of the Hansen Volubility parameters is well known to those skilled in 15 the art and is disclosed in particular in the article by C.M. Hansen: “The Three Dimensional Solubility Parameters”, J. Paint Technol. 39, 105 (1967), the entire contents of which are hereby incorporated by reference.

According to this Hansen space:

δ_{D} characterizes the London dispersion forces arising from the formation of dipoles induced during molecular impacts;

δₚ characterizes the Debye forces of interaction between permanent dipoles; and

δₕ characterizes the specific forces of interaction (such as hydrogen bonding, acid/base, donor/acceptor, etc.).

The parameters δ_{D}, δₚ and δₕ are generally expressed in (J/cm³)^{½.}

In the composition according to the invention, any non-volatile hydrocarbon-based oil or mixture of non-volatile hydrocarbon-based oils which satisfy the above relationships can be used. In this case, the solubility parameters of the mixture are determined from those of the non-volatile hydrocarbon based oils taken separately, according to the following relationships:

$${δ}_{Dmel} = {\sum }_{i} xi {δ}_{Di} ; {δ}_{ρ mel} = {\sum }_{i} xi {δ}_{ρ i} ; and {δ}_{hmel} = {\sum }_{i} xi {δ}_{hi}$$

where xi represents the volume fraction of the nonvolatile hydrocarbon-based oil in the mixture.

It is within the capabilities of a person skilled in the art to determine the amounts of each non-volatile hydrocarbon-based oil in order to obtain a mixture of non-volatile hydrocarbon-based oils which satisfies the above relationships.

The molecular mass of the hydrocarbon-based oils is preferably greater than 600 g/mol. More preferably, the molecular mass is greater than 700 g/mol, more particularly preferably greater than 900 g/mol, more especially preferably greater than 1000 g/mol, and most preferably greater than 1400 g/mol. These ranges include all values and subranges therebetween, including greater than or equal to 650, 750, 800, 850, 950, 1150, and 1200.

Preferabl examples of non-volatile hydrocarbon-based oils which may be mentioned are diusostearyl malate, monoesters or polyesters of polyols such as diglyceryl diisostearate or diglyceryl triisostearate or alternatively poly(12-hydroxy)stearic acids such as Solsperse 21 000 sold by the company Zeneca or Arlacel P 100 sold by the company Uniqema, and mixtures thereof. Diglyceryl diisostearate, diglyceryl triisostearate and poly(12-hydroxy)stearic acids and mixtures thereof are preferably used.

Their content by mass in the final composition is, for example, within the range from 3.5% to 40%, preferably from 4% to 30% and better still from 4% to 15%. These ranges include all values and subranges therebetween, including 3.75, 3.9, 4.1, 4.3, 4.6, 8, 11, 12, 18, 22, 25, 28, 32, 35, 38 and 39%.

As volatile hydrocarbon-based solvents which can be used in the invention, mention may be made of volatile solvents containing from 8 to 16 carbon atoms and mixtures thereof. In particular, these volatile solvents are chosen from branched C₈-C₁₆ alkanes, branched C8-C₁₆ esters and mixtures thereof. These solvents are preferably chosen from C₈-C₁₆ isoparaffins obtained in particular from petroleum, such as the “Isopar” and “Permetyl” products, isododecane or isohexadecane, isohexyl neopentanoate and mixtures thereof. Isododecane or isohexadecane or mixtures thereof are preferably used. The range of 8 to 16 carbon atoms includes all values and subranges therebetween, including 8-14 carbon atoms, 9, 10, 11, 12, 13, and 15 carbon atoms.

The volatile hydrocarbon-based solvent(s) according to the invention preferably represent(s) a content by mass of from 5% to 90%, preferably from 10% to 60% and better still from 20% to 50%. These ranges include all values and subranges therebetween, including 6, 8, 11, 18, 22, 25, 32, 37, 45, 55, 62, 68, 75, 85 and 88%.

The composition can also contain at least one additional fatty substance other than the non-volatile silicone compound, the volatile hydrocarbon-based solvent and the non-volatile hydrocarbon-based oil, chosen from waxes, gums and fatty substances that are pasty at room temperature, oils and mixtures thereof, of mineral, animal, plant or synthetic origin.

The additional oily fatty substances of the composition can be a cosmetically or dermatologically acceptable oil and in general a physiologically acceptable oil, chosen in particular from volatile or non-volatile oils of mineral, animal, plant or synthetic origin.

As additional oils which can be used in the composition according to the invention. mention may be made in particular of:

hydrocarbon-based oils of animal origin, such as perhydrosqualene;

hydrocarbon-based plant oils such as liquid triglycerides of fatty acids of 4 to 24 carbon atoms, such as heptanoic or octanoic acid triglycerides or alternatively, sunflower oil, corn oil, soybean oil, marrow oil, grapeseed oil, sesame or rape oil, hazelnut oil, apricot oil, macadamia oil, castor oil, avocado oil, caprylic/capric acid triglycerides such as those sold by the company Stearineries Dubois or those sold under the names Miglyol 810, 812 and 818 by the company Dynamit Nobel, jojoba oil or karite butter;

linear or branched hydrocarbons of mineral or synthetic origin, such as paraffin oils and derivatives thereof, petroleum jelly, polydecenes and hydrogenated polyisobutene, such as parleam;

synthetic esters and ethers in particular of fatty acids, such as the oils of formula R₃COOR₄ in which R₃ represents a higher fatty acid residue containing from 1 to 40 carbon atoms and R₄ represents a hydrocarbon based chain containing from 1 to 40 carbon atoms with R₃+R₄≧10, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisopropyl adipate, triisocetyl citrate, and fatty alkyl heptanoates, octanoates and decanoates; polyol esters such as propylene glycol dioctanoate, neopentyl glycol diheptanoate or diethylene glycol diisononanoate; and pentaerythritol esters such as pentaerythrityl tetraisostearate;

fatty alcohols containing from 12 to 26 carbon atoms, such as octyldodecanol, 2butyloctanol, 2-hexyldecanol, 2-undecylpentadecanol or oleyl alcohol;

partially hydrocarbon-based and/or silicone-based fluoro oils which are optionally volatile such as methoxynonafluorobutane;

volatile silicone oils such as volatile polydimethylsiloxanes (PDMSs) which are linear or cyclic and include from 2 to 7 silicon atoms, these silicones optionally including alkyl or alkoxy groups, pendant or at the end of a silicone chain, these groups containing from 1 to 10 carbon atoms, such as octamethylcyclotetrasiloxane, decamethylcyclopentasiloxane, hexadecamethylcyclohexasiloxane, heptamethylhexyltrisiloxane and heptamethylochyltrisilexane, and mixtures thereof;

mixtures thereof.

The non-volatile oil(s) of the composition can represent from 0.5% to 90% of the total weight of the composition and preferably from 8% to 75%. These ranges include all values and subranges therebetween, including 0.75, 1, 2, 3, 4, 5, 6, 12, 25, 32, 45, 55, 65, 73, 78, 85 and 88%.

Volatile oils are preferable for obtaining a film with “transfer-resistance” properties. After evaporation of these oils, a soft, homogeneous, glossy deposit is obtained which is comfortable on the skin or the lips, onto which the composition is applied. These volatile oils also make it easier to apply the composition to keratin materials.

These additional volatile silicone and fluoro oils in particular represent from 0% to 30% relative to the total weight of the composition and better still from 0% to 20%. These ranges include all values and subranges therebetween, including 0.1, 0.75, 1, 2, 3, 4, 5, 6, 12, 18, 22, 25 and 28%.

The composition of the invention can advantageously include one or more dyestuffs containing at least (one or more) pulverulent compounds and/or one or more liposoluble or water-soluble dyes, for example in a proportion of from 0% to 70% relative to the total weight of the composition and in particular from 0.01% to 70%. These ranges include all values and subranges therebetween, including 0.75, 1, 2, 3, 4, 5, 6, 12, 25, 32, 45, 55, 60, 65 and 68%. The pulverulent compound(s) may be chosen from the pigments, nacres and fillers usually used in cosmetic or dermatological compositions, and mixtures thereof. The pulverulent compounds advantageously represent from 0% to 50% (for example from 0.01% to 50%) relative to the total weight of the composition, and better still from 1% to 40%.

These ranges include all values and subranges therebetween, including 0.75, 1, 2, 3, 4, 5, 6, 12, 25, 32, 45, and 48%.

The pigments may be white or colored, mineral and/or organic, interferential or non-interferential, insoluble in the liquid fatty phase, and intended to color and/or opacity the composition. Among the mineral pigments which may be mentioned are titanium dioxide, optionally surface-treated. zirconium oxide, zinc oxide or cerium oxide, as well as zinc oxide, iron oxide or chromium oxide, manganese violet, ultramarine blue, chromium hydrate and ferric blue. Among the organic pigments which may be mentioned are carbon black, pigments of D & C type and lakes based on cochineal carmine or on barium, strontium, calcium or aluminum.

The nacreous pigments may be chosen from white nacreous pigments such as mica coated with titanium or with bismuth oxychloride, colored nacreous pigments such as titanium mica with iron oxides, titanium mica with, in particular, ferric blue or chromium oxide, titanium mica with an organic pigment of the abovementioned type, as well as nacreous pigments based on bismuth oxychloride.

The fillers may be mineral or organic, and lamellar or spherical. Mention may be made of talc, mica, silica, kaolin, Nylon® powder (Orgasol® from Atochem), poly-P-alanine powder and polyethylene powder, polytetrafluoroethylene (Teflon®) powders, lauroyllysine, starch, boron nitride, hollow microspheres such as those of polyvinylidene chloride acrylonitrile, for instance Expancel® (Nobel Industrie), acrylic acid copolymers, for instance Polygraph® (Dow Coming) and silicone resin microbeads (Tospearl® from Toshiba, for example), precipitated calcium carbonate, magnesium carbonate and hydrocarbonate, hydroxyapatite, hollow silica microspheres (Silica Beads® from Maprecos) and glass or ceramic microcapsules.

The liposoluble dyes are, for example, Sudan Red, DC Red 17, DC Green 6, β-carotene, soybean oil, Sudan Brown, DC Yellow 11, DC Violet 2, DC Orange 5 and quinoline yellow. They can represent from 0% to 20% and in particular 0.01% to 20% of the weight of the compositions and better still from 0.1% to 6%. These ranges include all values and subranges therebetween, including 0.75, 1, 2, 3, 4, 5, 12, 15, and 18%. The water-soluble dyes are, for example, beetroot juice or methylene blue and can represent up to 6% of the total weight of the composition.

The composition of the invention can also contain one or more cosmetic or dermatological active agents such as those used conventionally.

As cosmetic or dermatological active agents which can be used in the composition of the invention, mention may be made of moisturizers, vitamins, essential fatty acids, sphingolipids and sunscreens. These active agents are used in an amount which is usual for those skilled in the art and in particular at concentrations of from 0% to 20% and in particular from 0.001% to 20% relative to the total weight of the composition. These ranges include all values and subranges therebetween, including 0.75, 1, 2, 3, 4, 5, 6, 10, 12, 15 and 18%.

Depending on the type of application envisaged, the composition according to the invention can furthermore include the constituents conventionally used in the fields under consideration, which are present in an amount which is suitable for the desired presentation form.

Among the waxes that are solid at room temperature, which may be present in the composition according to the invention, mention may be made of hydrocarbon-based waxes such as optionally modified beeswax, camauba wax, candelilla wax, ouricury wax, Japan wax, cork fibre wax, sugar cane wax, paraffin wax, lignite wax, microcrystalline waxes, lanolin wax, montan wax, ozokerites, polyethylene waxes, the waxes obtained by Fischer-Tropsch synthesis and C₂₀-C₆₀ fatty alcohols. It is also possible to use silicone waxes, among which mention may be made of alkyl- and alkoxypolymethylsiloxanes and/or polymethylsiloxane esters, and mixtures thereof.

The waxes may be present in a proportion of 0-50% (for example from 0.01% to 50%) by weight in the composition and better still from 5% to 20%, so as not to excessively reduce the gloss of the composition and of the film deposited on the lips and/or the skin. These ranges include all values and subranges therebetween, including 0.75, 1, 2, 3, 4, 6, 8, 12, 25, 32, 38, 45, and 48%.

Pasty fatty substances which may be mentioned are fatty substances with a melting point ranging from 25° C. to 45° C. and/or a viscosity at 40° C. ranging from 0.1 Pa.s to 40 Pa.s, measured using a Contraves TV viscometer equipped with an MS-r3 or MS-r4 spindle spinning at 60 Hz. Examples of pasty fatty substances which may be mentioned are PDMSs with pendant chains of the alkyl or alkoxy type containing from 8 to 24 carbon atoms, for instance stearyldimethicone: esters of fatty alcohol or of fatty acid, for instance cholesterol esters, polyvinyl laurate, arachidyl propionate; PVP/eicosene copolymer; lanolins and derivatives thereof such as acetylated lanolins or oxypropylenated lanolins, and mixtures thereof.

The nature and amount of the waxes, pasty fatty substances and gums depend on the desired mechanical properties and textures.

The composition can also include any additive usually used in such compositions, such as thickeners, antioxidants, fragrances, preserving agents, surfactants, liposoluble polymers, for instance polyalkylenes, in particular polybutene, polyacrylates and silicone polymers that are compatible with the fatty phase, as well as polyvinylpyrrolidone derivatives. Needless to say, a person skilled in the art will take care to select this or these optional additional compound(s), and/or the amount thereof, such that the advantageous properties of the composition according to the invention are not, or are not substantially, adversely affected by the envisaged addition.

In one preferred embodiment of the invention, the compositions according to the invention can be prepared in the usual manner by a person skilled in the art. They can be in the form of a cast product and, for example, in the form of a stick or tube, or in the form of a dish which can be used by direct contact or with a sponge or alternatively in a boiling pan. In particular, they find an application as cast foundations, cast blushers or eye shadows, lipsticks, care bases or care balms for the lips and concealer products. They can also be in the form of a soft paste or alternatively a gel or a more or less fluid cream. In this case, they can constitute foundations or lipsticks, lip glosses, suncare products or skin coloring products.

The compositions of the invention preferably contain a continuous fatty (or lipophilic) phase and are preferably anhydrous or can contain less than 5% by weight of water relative to the total weight of the composition. In this case, they can be in particular in the form of an oily gel, an oily liquid, a paste or a stick or alternatively in the form of a vesicular dispersion containing ionic and/or nonionic lipids. They can also be in the form of a simple or multiple emulsion containing an oily or aqueous continuous phase, or an oily dispersion in an aqueous phase with of vesicles containing ionic and/or nonionic lipids. These presentation forms are prepared according to the usual methods of the fields under consideration.

The composition is preferably a lipstick including at least one volatile hydrocarbon-based solvent, at least one non-volatile silicone compound, which is soluble or dispersible in the volatile hydrocarbon-based solvent and at least one non-volatile hydrocarbon-based oil which is soluble in the volatile solvent and incompatible with the non-volatile silicone compound, the non-volatile hydrocarbon-based oil having solubility parameters such that 16.40 (J/cm³)^{½}≦δ_{D}≦19.00 (J/cm³)^{½} and 2.00 (J/cm³)^{½}≦δₐ≦9.08 (J/cm³)^{½}, the solvent, the silicone compound and the hydrocarbon based oil being as defined above.

These compositions for topical application can preferably constitute a cosmetic or dermatological protective, treatment or care composition for the face, for the neck, for the hands or for the body (for example a care cream, anti-sun oil or body gel), a make-up composition (for example a make-up gel, cream or stick) or an artificial tanning composition or skin-protecting composition.

### EXAMPLES

Having generally described this invention, a further understanding can be obtained by reference to certain specific examples which are provided herein for purposes of illustration only and are not intended to be limiting unless otherwise specified. The percentages are percentages by weight.

### Examples 1 and 2

Lipsticks in the Form of “gloss” in a Boiling Pan

The formulations below were compared:

| | Example 1 | Example 2 |
|---|---|---|
| Phase A | | |
| Hydrogenated polyisobutene | 11.15% | 11.15% |
| Poly(l2-hydroxy)stearic acid sold | 4.86% | — |
| under the reference Solsperse 21000 | | |
| by the company Zeneca | | |
| Preserving agent | qsp | qsp |
| Antioxidant | qsp | qsp |
| Phase B | | |
| Microcrystalline wax | 7.63% | 7.63% |
| Ozokerite | 5.27% | 5.27% |
| Phase C | | |
| Titanium oxide | 0.38% | 0.38% |
| DC Red No. 7 | 1.38% | 1.38% |
| FDC yellow No. 6 Al lake | 2.94% | 2.94% |
| Kaolin | 7.50% | 7.50% |
| Phase D | | |
| Phenyltrimethicone (1000 cSt) | 36.35% | 41.21% |
| Phase E | | |
| Isododecane | 22.08% | 22.08% |
| | 100% | 100% |
| | by mass | by mass |

Procedure

The particulate phase C is ground in phase A using a three-roll mill. The waxy phase B is then added and the mixture is heated at 100° C. until the waxes have completely dissolved. The phenyltrimethicone is added at 100° C., followed by the isododecane at 80-90° C. After homogenization, the composition can be introduced at 60-80° C. into suitable boiling pans. Example 1 has better gloss and transfer-resistance properties than those of Example 2.

### Example No. 3

Lipstick in the Form of a “gloss” in a Boiling Pan

| | |
|---|---|
| Phase A | |
| Hydrogenated polyisobutene | 11.15% |
| Diglyceryl diisostearate sold under | 4.86% |
| the reference Salacos 42 by the | |
| company Nisshin Oil Mills | |
| Preserving agent | qsp |
| Antioxidant | qsp |
| Phase B | |
| Microcrystalline wax | 7.63% |
| Ozokerite | 5.27% |
| Phase C | |
| Titanium oxide | 0.38% |
| DC Red No. 7 | 1.38% |
| DC yellow No. 6 Al lake | 2.94% |
| Kaolin | 7.50% |
| Phase D | |
| Phenyltrimethicone (1000 cSt) | 36.35% |
| Phase E | |
| Isododecane | 22.08% |
| | 100% by mass |

### Example No. 4

Lipstick in the Form of a “gloss” in a Boiling Pan

| | |
|---|---|
| Phase A | |
| Hydrogenated polyisobutene | 10.95% |
| Poly(12-hydroxy)stearic acid sold | 4.86% |
| under the reference Solsperse 21000 | |
| by the company Zeneca | |
| α-Bisabolol | 0.2% |
| PVP/eicosene copolymer | 5.55% |
| Preserving agent | qsp |
| Antioxidant | qsp |
| Phase B | |
| Microcrystalline wax | 4.35% |
| Ozokerite | 3.00% |
| Phase C | |
| Titanium oxide | 0.28% |
| DC Red No. 7 | 1.03% |
| FDC yellow No. 6 Al lake | 2.19% |
| Kaolin | 7.00% |
| Acrylates copolymer (Polytrap ®) | 0.5% |
| Phase D | |
| Phenyltrimethicone (1000 cSt) | 36.35% |
| Phase E | |
| Mica | 1.2% |
| Phase F | |
| Isododecane | 22.08% |
| | 100% by mass |

### Example No. 5

Lipstick in Stick Form

| | |
|---|---|
| Phase A | |
| Hydrogenated polyisobutene | 11.00% |
| Diglyceryl diisostearate sold under | 4.86% |
| the reference Salacos 42 by the | |
| company Nisshin Oil Mills | |
| PVP/eicosene copolymer | 5.00% |
| Antioxidant | qsp |
| Phase B | |
| Polyethylene wax (Mn* = 400) | 10.00% |
| Phase C | |
| Titanium oxide | 0.28% |
| DC Red No. 7 | 1.03% |
| FDC yellow No. 6 Al lake | 2.19% |
| Kaolin | 7.50% |
| Phase D | |
| Phenyltrimethicone (1000 cSt) | 35.00% |
| Phase E | |
| Mica | 1.00% |
| Phase F | |
| Isododecane | 22.08% |
| | 100% by mass |

*Mn = number-average molecular mass

Procedure

The particulate phase C is ground in phase A using a three-roll mill. The polyethylene wax is then added and the mixture is heated at 100° C. until the wax has completely dissolved. The phenyltrimethicone and the mica are then added at 100° C., followed by the isododecane at 80-90° C. The mixture can then be cast at 80-85° C. into suitable molds in order to obtain sticks.

The composition of Example 4 is tested in comparison with a commercial formulation of liquid lipstick, “liquid lip” from Revlon not containing the combination according to the invention of non-volatile silicone compound, of volatile hydrocarbon-based solvent and of non-volatile hydrocarbon-based oil.

The composition of the invention was considered as having a glossier and more fluid appearance than that of the prior art.

The composition of the invention was considered as being more glossy when applied and over time, as well as much more comfortable.

The two lipsticks do not stick when applied. The staying power over time was considered as being identical; they undergo little transfer, stay in place well and thus require a removal operation. They wear away uniformly over time.

### Example No. 6

Lipstick in Stick Form

| | |
|---|---|
| Phase A | |
| Hydrogenated polyisobutene | 14.36% |
| Poly(12-hydroxy)stearic acid sold | 4.00% |
| under the reference Solsperse 21000 | |
| by the company Zeneca | |
| PVP/eicosene copolymer | 5.00% |
| α-Bisabolol | 0.40% |
| Antioxidant | qsp |
| Phase B | |
| Carnauba wax | 2.02% |
| Ozokerite | 2.40% |
| Polyethylene wax (Mn* = 650) | 8.08% |
| Phase C | |
| Titanium oxide | 1.80% |
| DC Red No. 7 Ca lake | 2.90% |
| FDC yellow No. 6 Al lake | 3.30% |
| DC Red No. 21 Al lake | 0.60% |
| Black iron oxide | 0.06% |
| Kaolin | 1.84% |
| Phase D | |
| Phenyltrimethicone (1000 cSt) | 34.32% |
| Phase E | |
| Mica | 1.14% |
| Phase F | |
| Isododecane | 17.72% |
| | 100% by mass |

*Mn = number-average molecular mass

Procedure The particulate phase C is ground in phase A using a three-roll mill. The waxy phase B is then added and the mixture is heated at 105° C. until the waxes have completely dissolved. The phenyltrimethicone and the mica are then added at 105° C., followed by the isododecane at 90-100° C. The resulting mixture can then be cast at 90-1 00° C. in suitable molds to obtain sticks with good staying power, which are not sticky, do not transfer and are pleasant to wear.

### Example No. 7

Lipstick in Stick Form

| | |
|---|---|
| Phase A | |
| Hydrogenated polyisobutene | 13.82% |
| Diglyceryl triisostearate sold under | 3.85% |
| the reference Salacos 43 by the | |
| company Nisshin Oil Mills | |
| PVP/eicosene copolymer | 4.82% |
| α-Bisabolol | 0.40% |
| Antioxidant | qsp |
| Phase B | |
| Ozokerite | 3.40% |
| Polyethylene wax (Mn* = 500) | 11.60% |
| Phase C | |
| DC Red No. 7 | 2.72% |
| FDC Blue No. 1 Al lake | 0.81% |
| Brown iron oxide | 1.01% |
| Black iron oxide | 2.01% |
| Kaolin | 3.94% |
| Phase D | |
| Phenyltrimethicone (1000 cSt) | 24.11% |
| Phase E | |
| Nacres | 5.45% |
| Phase F | |
| Isododecane | 22.00% |
| | 100% by mass |

*Mn = number-average molecular mass

The procedure is identical to that of Example 6. The cosmetic properties of the stick are identical to those of the stick of Example 6.

### Example 8

Lipstick in Stick Form

| | |
|---|---|
| Phase A | |
| Poly (12-hydroxy)stearic acid sold | 4.00% |
| under the reference Solsperse 21000 | |
| by the company Zeneca | |
| PVP/eicosene copolymer | 3.00% |
| Arachidyl propionate | 5.00% |
| α-Bisabolol | 0.40% |
| Antioxidant | qsp |
| Phase B | |
| Hydrogenated polyisobutene | 18.76% |
| Bentone | 0.54% |
| Phase C | |
| Ozokerite | 3.78% |
| Polyethylene wax (Mn* = 650) | 11.22% |
| Phase D | |
| Titanium oxide | 1.80% |
| 3C Red No. 7 Ca lake | 2.90% |
| FDC Yellow No. 6 Al lake | 3.30% |
| DC Red No. 21 Al lake | 0.60% |
| slack iron oxide | 0.06% |
| Kaolin | 1.84% |
| Phase E | |
| Phenyltrimethicone (1000 cSt) | 21.60% |
| Phase F | |
| Mica | 1.14% |
| Phase G | |
| Isohexadecane | 20.00% |
| | 100% by mass |

*Mn = number-average molecular mass

Procedure

Phase B is prepared by dispersing the bentone in the hydrogenated polyisobutene. The particulate phase D is ground in phase A using a three-roll mill. Phase B is then added, followed by the waxy phase C. The mixture is heated at 105° C. until the waxes have completely dissolved. The phenyltrimethicone and the mica are then added at 105° C., followed by the isohexadecane at 90-100° C. The composition can then be cast at 90-100° C. in suitable molds to obtain sticks which have identical properties to the stick of Example 6.

This application is based on French patent application No. 9915373, filed Dec. 3, 1999, the entire contents of which are hereby incorporated by reference, the same as if set forth at length.

Having now fully described this invention, it will be apparent to one of ordinary skill in the art that many changes and modifications can be made thereto without departing from the spirit or scope of the invention as set forth herein.

1. A composition, comprising:
at least one volatile hydrocarbon-based solvent;
at least one non-volatile silicone compound which is soluble or dispersible in said volatile hydrocarbon-based solvent; and
at least one non-volatile hydrocarbon-based oil which is soluble in said volatile hydrocarbon-based solvent and incompatible with said non-volatile silicone compound;
wherein said non-volatile hydrocarbon-based oil has solubility parameters satisfying the relationships, 16.40 (J/cm³)^{½}≦δ_{D}<19.00 (J/cm³)^{½} and 2.00 (J/cm³)^{½}≦δₐ≦9.08 (J/cm³)^{½}.

2. The composition according to claim 1, wherein said hydrocarbon-based oil has solubility parameters satisfying the relationship, 16.70 (J/cm³)^{½}≦δ_{D}≦18.50 (J/cm³)^{½}.

3. The composition according to claim 1, wherein said hydrocarbon-based oil has solubility parameters satisfying the relationship, 4.00 (J/cm³)^{½}≦δₐ≦9.08 (J/cm³)^{½}.

4. The composition according to claim 1, wherein said hydrocarbon-based oil has a molar mass of greater than 600 g/mol.

5. The composition according to claim 1, wherein said hydrocarbon-based oil has a chemical structure comprising at least one nonionic polar group selected from the group consisting of —COOH; —OH; —PO₄; —NHR with R representing H or a linear or branched C₁ to C₂₀ alkyl or alkoxy group; —NR₁R₂ with R₁ and R₂ optionally forming a ring and each independently representing a linear or branched C₁ to C₂₀ alkyl or alkoxy group or radical; and 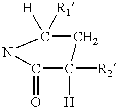
with R₁′ and R₂′ each independently representing H or a linear or branched C₁ to C₂₀ alkyl or alkoxy group; and combinations thereof.

6. The composition according to claim 1, wherein said hydrocarbon-based oil has a chemical structure comprising at least two nonionic polar groups selected from the group consisting of —COOH; —OH; —PO₄; and 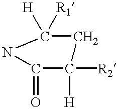
with R₁′ and R₂′ each independently representing H or a linear or branched C₁ to C₂₀ alkyl or alkoxy group; and combinations thereof.

7. The composition according to claim 1, wherein said non-volatile hydrocarbon-based oil is selected from the group consisting of duisostearyl malate, polyol monoester, polyol polyester, poly(12-hydroxy)stearic acid, and mixtures thereof.

8. The composition according to claim 1, wherein said non-volatile hydrocarbon-based oil is selected from the group consisting of diglyceryl duisostearate, diglyceryl truisostearate, poly(12-hydroxy)stearic acid, and mixtures thereof.

9. The composition according to claim 1, wherein said non-volatile hydrocarbon-based oil is present in an amount ranging from 3.5% to 40% by mass, based on the total mass of the composition.

10. The composition according to claim 1, wherein said non-volatile silicone compound is selected from the group consisting of silicone-containing compounds that are liquid at room temperature, and mixtures thereof.

11. The composition according to claim 1, wherein said non-volatile silicone compound has a viscosity ranging from 5 to 10,000 cSt.

12. The composition according to claim 1, wherein said non-volatile silicone compound is selected from the group consisting of non-volatile polydimethylsiloxanes (PDMSs); polydimethylsiloxanes comprising one or more C₂-24 alkyl, alkoxy or phenol groups that are pendant or at the end of a silicone chain; phenyl trimethicones; phenyl dimethicones; phenyl trimethylsiloxydiphenylsiloxanes; diphenyl dimethicones; diphenyl methyldiphenyltrisiloxanes; 2-phenylethyl trimethylsiloxysilicates; fluorosilicones comprising one or more C₁₋₁₂ fluoro groups that are pendant or at an end of the silicone chain, at least one, more than one, or all of hydrogen atoms in said C₁₋₁₂ fluoro groups being substituted with fluorine atoms; silicone resins; and mixtures thereof.

13. The composition according to claim 1, wherein said non-volatile silicone compound is present in an amount ranging from 0.5% to 90% by mass, based on the total mass of the composition.

14. The composition according to claim 1, further comprising at least one selected from the group consisting of cosmetic active agents, dermatological active agents, dyestuffs, and combinations thereof.

15. The composition according to claim 1, wherein said volatile hydrocarbon-based solvent is at least one selected from the group consisting of volatile solvents containing from 8 to 14 carbon atoms, and mixtures thereof.

16. The composition according to claim 1, wherein said volatile hydrocarbon-based solvent is at least one selected from the group consisting of branched C₈-C₁₄ alkane, branched C₈-C₁₄ ester, and mixtures thereof.

17. The composition according to claim 1, wherein said volatile hydrocarbon-based solvent is at least one selected from the group consisting of C₈-C₁₄ isoparaffin, isododecane, isohexadecane, and mixtures thereof.

18. The composition according to claim 1, wherein said volatile hydrocarbon-based solvent is selected from the group consisting of isododecane, isohexadecane, and mixtures thereof.

19. The composition according to claim 1, wherein said volatile hydrocarbon-based solvent is present in an amount ranging from 5% to 90% by mass, based on the total mass of the composition.

20. The composition according to claim 1, wherein a ratio by mass of said non-volatile silicone compound relative to said non-volatile hydrocarbon-based oil is greater than or equal to 1.

21. The composition according to claim 1, further comprising at least one fatty substance other than said non-volatile silicone compound, said volatile hydrocarbon-based solvent and said non-volatile hydrocarbon-based oil, said fatty substance being selected from the group consisting of waxes, gums, fatty substances that are pasty at room temperature, oils, and mixtures thereof.

22. The composition according to claim 1, further comprising one or more dyestuff, wherein said dyestuff comprises at least one pulverulent compound selected from the group consisting of fillers, pigments, nacres, and mixtures thereof.

23. The composition according to claim 22, wherein said pulverulent compound represents up to 50% of the total weight of the composition.

24. The composition according to claim 1, which is in the form of at least one selected from the group consisting of stick, tube, soft paste, dish, oily gel, oily liquid, vesicular dispersion comprising ionic and/or nonionic lipids, water-in-oil, oil-in-water emulsion, and combinations thereof.

25. The composition according to claim 1, which is in anhydrous form.

26. The composition according to claim 1, which is in the form of at least one selected from the group consisting of foundation, blusher, eyeshadow, lipstick, care base or care balm for the lips, concealer product, eyeliner, mascara, and combinations thereof.

27. A composition selected from the group consisting of lipstick composition, cosmetic composition, transfer-resistant composition, glossy composition, composition for topical application, and combinations thereof, comprising:
at least one volatile hydrocarbon-based solvent;
at least one non-volatile silicone compound which is soluble or dispersible in said volatile hydrocarbon-based solvent; and
at least one non-volatile hydrocarbon-based oil which is soluble in said volatile hydrocarbon-based solvent and incompatible with said non-volatile silicone compound;
wherein said non-volatile hydrocarbon-based oil has solubility parameters satisfying the relationships, 16.40 (J/cm³)^{½}≦δ_{D}≦19.00 (J/cm³)^{½} and 2.00 (J/cm³)^{½}≦δₐ≦9.08 (J/cm³)^{½}.

28. A method for preparing a composition, comprising:
contacting
at least one volatile hydrocarbon-based solvent;
at least one non-volatile silicone compound which is soluble or dispersible in said volatile hydrocarbon-based solvent; and
at least one non-volatile hydrocarbon-based oil which is soluble in said volatile hydrocarbon-based solvent and incompatible with said non-volatile silicone compound;
wherein said non-volatile hydrocarbon-based oil has solubility parameters satisfying the relationships, 16.40 (J/cm³)^{½}≦δ_{D}≦19.00 (J/cm³)^{½} and 2.00 (J/cm³)^{½}≦δₐ≦9.08 (J/cm³)^{½}.

29. A method of use, comprising applying the composition according to claim 1 to the lips or the skin, respectively.

30. A process selected from the group consisting of reducing or preventing the transfer of a film of composition deposited on the skin and/or the lips of a human being to a support placed in contact with the film, preserving the gloss of a film of a composition deposited on the skin and/or lips of a human being, reducing or preventing the transfer of a care or make-up composition for the skin or the lips onto a support other than the skin or the lips, and combinations thereof, said composition comprising at least one ingredient selected from the group consisting of cosmetic or dermatological active agents, dyestuffs, and combinations thereof, which process comprises introducing into said composition:
at least one volatile hydrocarbon-based solvent;
at least one non-volatile silicone compound which is soluble or dispersible in said volatile hydrocarbon-based solvent; and
at least one non-volatile hydrocarbon-based oil which is soluble in said volatile hydrocarbon-based solvent and incompatible with said non-volatile silicone compound;
wherein said non-volatile hydrocarbon-based oil has solubility parameters satisfying the relationships, 16.40 (J/cm³)^{½}≦δ_{D}≦19.00 (J/cm³)^{½} and 2.00 (J/cm³)^{½}≦δₐ≦9.08 (J/cm³)^{½}.
